(19) United States
(12) Patent Application Publication
(10) Pub. No.: US 2021/0183488 A1
(43) Pub. Date: Jun. 17, 2021

(54) DEVICES, SYSTEMS AND METHODS FOR INTENTIONAL SENSING OF ENVIRONMENTAL CONDITIONS

(71) Applicant: Colorado State University Research Foundation, Fort Collins, CO (US)

(72) Inventors: Bradley Reisfeld, Fort Collins, CO (US); Steve Simske, Fort Collins, CO (US); Wes Anderson, Fort Collins, CO (US); Doreen E. Martinez, Fort Collins, CO (US)

(21) Appl. No.: 17/121,147

(22) Filed: Dec. 14, 2020

Related U.S. Application Data
(60) Provisional application No. 62/947,956, filed on Dec. 13, 2019.

(51) Int. Cl. G16H 20/10; G06N 20/10; G16H 10/60; G16H 30/40; G16H 40/40; G06K 9/00

(52) U.S. Cl. CPC G16H 20/10; G06N 20/10; G16H 10/60; G06K 9/00335; G16H 40/40; G06K 9/00013; G16H 30/40

(57) ABSTRACT

Described herein are systems, methods and devices for measuring environmental conditions. An example system includes an appliance including a housing, a first sensor, and a second sensor configured to measure a property of a sample, where the first and second sensors are attached to or arranged within the housing. The system also includes a computing device in operable communication with the appliance. The computing device includes a processor and a memory, the memory having computer-executable instructions stored thereon that, when executed by the processor, cause the processor to: receive a first signal from the first sensor; analyze the first signal to determine an identity and an intent of a user; and initiate an action using the second sensor based on the intent of the user.

### CROSS-REFERENCE TO RELATED APPLICATIONS

This application claims the benefit of U.S. provisional patent application No. 62/947,956, filed on Dec. 13, 2019, and titled “Magic Wand Appliance to Help Engage Popular Epidemiology,” the disclosure of which is expressly incorporated herein by reference in its entirety.

### BACKGROUND

Increasing availability and advances in monitoring technologies and the increasing popularity of mobile devices, social media, and cloud-based information sharing, create a growing opportunity for individuals to perform environmental measurements. This can benefit users and communities by allowing them to identify and mitigate air quality issues (e.g. pollution) and reduce the effects associated with the same. For example, it can be beneficial for individuals to participate in identifying and mitigating pollution and associated solute- and particulate-engendered local health and illness patterns. Solutions for increasing access to environmental measurements can benefit from simple user interfaces and user authentication systems.

Therefore, what is needed are systems, appliances, and methods for performing environmental measurements, including systems, devices and methods for performing intentional environmental measurements.

### SUMMARY

An example system for measuring environmental conditions is described herein. The system includes an appliance including: a housing, a first sensor, and a second sensor configured to measure a property of a sample, where the first and second sensors are attached to or arranged within the housing. The system also includes a computing device in operable communication with the appliance, where the computing device includes a processor and a memory, the memory having computer-executable instructions stored thereon that, when executed by the processor, cause the processor to: receive a first signal from the first sensor; analyze the first signal to determine an identity and an intent of a user; and initiate an action using the second sensor based on the intent of the user.

Alternatively or additionally, the first sensor is a sensor configured to collect data suitable for biometrics. Optionally, the sensor configured to collect data suitable for biometrics includes at least one of a camera, a fingerprint sensor, a microphone, an accelerometer, a strain gauge, an acoustic sensor, a temperature sensor, or a hygrometer.

Alternatively or additionally, the first sensor is an orientation sensor. Optionally, the orientation sensor includes at least one of a gyroscope, an accelerometer, or a magnetometer.

Alternatively or additionally, the second sensor is a consumable sensor.

Alternatively or additionally, the second sensor is at least one of a Surface-Enhanced Raman Spectroscopy (SERS) sensor, an analyte sensor, a magnetoencephalography sensor, an impedance plethysmography sensor, a plurality of electrodes, a strain gauge, a thermistor, a linear variable differential transformer (LVDT), a capacitance sensor, or an acoustic sensor.

Alternatively or additionally, the sample is a solid, a liquid, or a gas.

Alternatively or additionally, the memory has further computer-executable instructions stored thereon that, when executed by the processor, cause the processor to receive a second signal from the second sensor.

In some implementations, the appliance further includes a dispensing unit configured to dispense a dosage of a medicine or an amount of reagent, where the dispensing unit is attached to or arranged within the housing. Optionally, the memory has further computer-executable instructions stored thereon that, when executed by the processor, cause the processor to: receive a second signal from the second sensor; and dispense the dosage of the medicine or the amount of reagent in response to the second signal. Optionally, the dispensing unit includes a locking mechanism.

Alternatively or additionally, the first signal includes movement data. Optionally, the movement data includes a plurality of anatomic movements. In some implementations, the movement data includes at least one of acceleration, angular velocity, or heading information. Optionally, analyzing the first signal to determine an identity and an intent of a user includes applying a gesture algorithm to the first signal. In some implementations, the gesture algorithm is a Dynamic Time Warping (DTW) algorithm, a Hidden Markov Model (HMM) algorithm, or a Support Vector Machine (SVM).

Alternatively or additionally, the housing is an elongated cylinder.

Alternatively or additionally, the housing includes a plurality of modular sections, each of the first sensor and the second sensor is attached to or arranged within a respective modular section housing. Optionally, the respective modular section that houses the second sensor is configured to store the sample. In some implementations, the respective modular section that houses the second sensor is further configured to contain a reaction involving the sample.

Alternatively or additionally, the system includes a wireless transceiver configured to operably couple the appliance and the computing device.

Alternatively or additionally, the appliance further includes a location sensor.

An example appliance for measuring environmental conditions is also described herein. The appliance includes a housing, a first sensor, a second sensor configured to measure a material property of a sample, and a wireless transceiver in operable communication with the first sensor and the second sensor, where the wireless transceiver is configured to operably couple with a remote computing device, and where the first sensor, the second sensor, and the wireless transceiver are attached to or arranged within the housing.

Alternatively or additionally, the wireless transceiver is a low-power wireless transceiver. Optionally, the first sensor is a sensor configured to collect data suitable for biometrics. In some implementations the sensor configured to collect data suitable for biometrics includes at least one of a camera, a fingerprint sensor, a microphone, an accelerometer, a strain gauge, an acoustic sensor, a temperature sensor, or a hygrometer. Optionally, the first sensor is an orientation sensor. Optionally, the orientation sensor includes at least one of a gyroscope, an accelerometer, or a magnetometer. In some implementations, the second sensor is a consumable sensor. Optionally, the second sensor is at least one of a Surface-Enhanced Raman Spectroscopy (SERS) sensor, an analyte sensor, a magnetoencephalography sensor, an impedance plethysmography sensor, a plurality of electrodes, a strain gauge, a thermistor, a linear variable differential transformer (LVDT), a capacitance sensor, or an acoustic sensor. In some implementations, the sample is a solid, a liquid, or a gas. Optionally, the appliance further includes a dispensing unit configured to dispense a dosage of a medicine or an amount of reagent, where the dispensing unit is attached to or arranged within the housing. In some implementations, the dispensing unit includes a locking mechanism.

Optionally, the housing is an elongated cylinder. In some implementations, the housing includes a plurality of modular sections, where each of the first sensor and the second sensor is attached to or arranged within a respective modular section housing. Optionally, the respective modular section that houses the second sensor is configured to store the sample. In some implementations, the respective modular section that houses the second sensor is further configured to contain a reaction involving the sample.

Optionally, the appliance further includes a location sensor.

An example method for measuring an environmental condition is also is described herein. The method can include receiving a first signal from an appliance, the appliance being configured to measure an environmental condition; analyzing the first signal to determine an identity and an intent of a user; initiating an environmental measurement of an environmental sample based on the intent of the user; and receiving a second signal from the appliance, the second signal including information related to the environmental measurement.

In some implementations, the method can optionally further include acquiring the environmental sample, where the environmental sample includes at least one of a solid, liquid or gas.

Alternatively or additionally, the method can optionally further include dispensing a dosage of a medicine or an amount of reagent in response to the second signal.

In some implementations, the first sensor is a sensor configured to collect data suitable for biometrics. Alternatively or additionally, the first sensor is an orientation sensor. In some implementation, the second sensor is a consumable sensor. Alternatively or additionally, the second sensor is at least one of a Surface-Enhanced Raman Spectroscopy (SERS) sensor, an analyte sensor, a magnetoencephalography sensor, an impedance plethysmography sensor, a plurality of electrodes, a strain gauge, a thermistor, a linear variable differential transformer (LVDT), a capacitance sensor, or an acoustic sensor.

In some implementations, the first signal includes movement data. Alternatively or additionally, the movement data includes a plurality of anatomic movements. Alternatively or additionally, the movement data includes at least one of acceleration, angular velocity, or heading information. Alternatively or additionally, analyzing the first signal to determine an identity and an intent of a user includes applying a gesture algorithm to the first signal. Optionally, the gesture algorithm is a Dynamic Time Warping (DTW) algorithm, a Hidden Markov Model (HMM) algorithm, or a Support Vector Machine (SVM).

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flowchart illustrating a method of performing an environmental measurement based on the intent and identity of a user according to implementations described herein.

FIG. 2 is an architecture diagram, according to one implementation described herein.

FIGS. 3A-3C are illustrations of implementations of the present disclosure, where FIG. 3A illustrates an implementation of the present disclosure shaped as a “wand;” FIG. 3B illustrates an implementation of the present disclosure built into the side of a mobile phone; and FIG. 3C illustrates clips that can be used to activate the sensors shown in FIG. 3B.

FIG. 4 illustrates a LSM9DS1 9-axis accelerometer/gyroscope/magnetometer attached to the end of a 6-in long PVC pipe (“wand”) as part of an experiment described herein.

FIG. 5 illustrates types of translational movements within a reference frame where (a) denotes movement in the x-direction, (b) denotes movement in the y-direction, and (c) denotes a movement in the z-direction. The directions are based on the positioning of the LSM9DS1 on the end of the wand shown in FIG. 4;” the x, y, and z-directions are displayed.

FIG. 6 illustrates types of rotational movements where (d) denotes movement in the combined y- and z-directions; (e) denotes movement in the combined x- and z-directions; and denotes (f) a movement in the combined x- and y-directions. The directions are based on the positioning of the LSM9DS1 on the end of the wand shown in FIG. 4; the x, y, and z-directions from FIG. 5 are also illustrated.

FIG. 7 illustrates a flowchart of a method for optimizing the threshold and weight of the accelerometer and gyroscope data, respectively. C1 and C2 represent the optimized thresholds for the accelerometer and gyroscope data, respectively.

FIG. 8 is a table of experimental results from applying a meta-algorithmic classifier for translational and rotational movement according to one implementation of the present disclosure.

FIG. 9 is a table of experimental results from a “test” set made within 30 degrees of each axis for both left and right-handed gestures (from 25 total gestures).

FIG. 10 is an example of mapping of data before and after axis projection was applied to the data. The circles represent the original mapping, and the “x” marks represent the mapping of the shifted data. The line of best fit for each data set is also illustrated.

FIG. 11 is a table of experimental results illustrating the effects of an axis shift on the translational data, where the boldfaced data is the axis in which the movement was supposed to have been made.

FIGS. 12A-12B are confusion matrices, where FIG. 12A represents a confusion matrix for left-handed translational movements and FIG. 12B represents a confusion matrix for right-handed translational movements.

FIG. 13 is a table of experimental results illustrating accuracy ranges of the translational movements before and after applying an axis shift to those movements.

FIG. 14 is a table of experimental results illustrating the mean increase of the data toward the correct axis for -, y-, and z-movements, respectively.

FIG. 15 illustrates an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for performing certain measurements (e.g. concentrations of pollutants), it will become evident to those skilled in the art that the implementations are not limited thereto, but are applicable to performing any kind of environmental measurement.

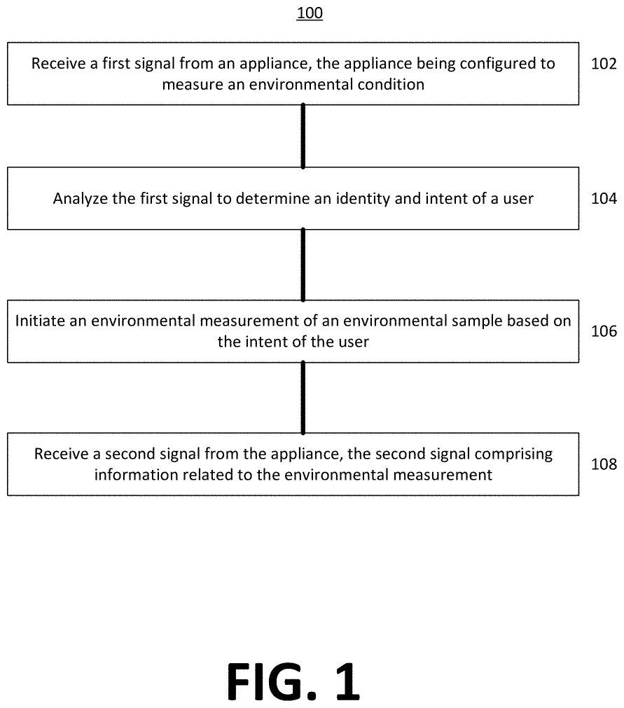

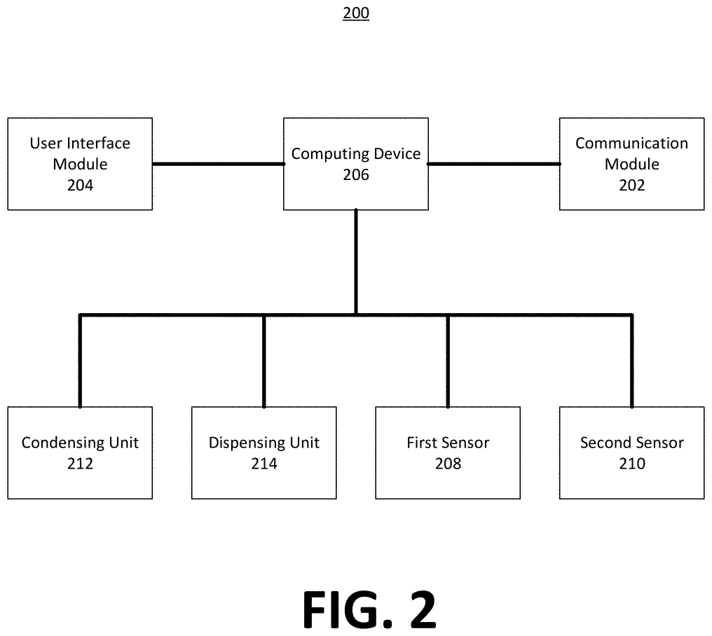

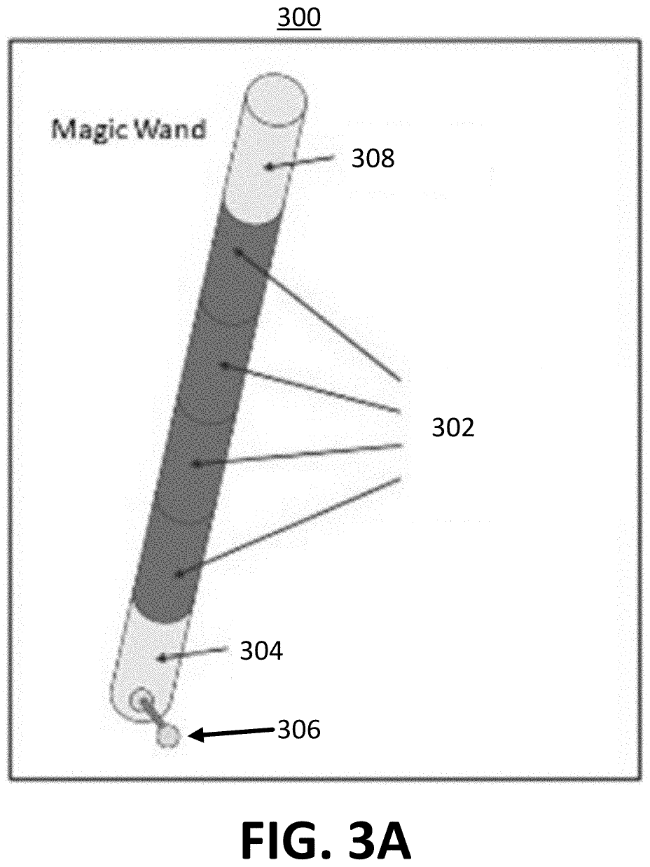

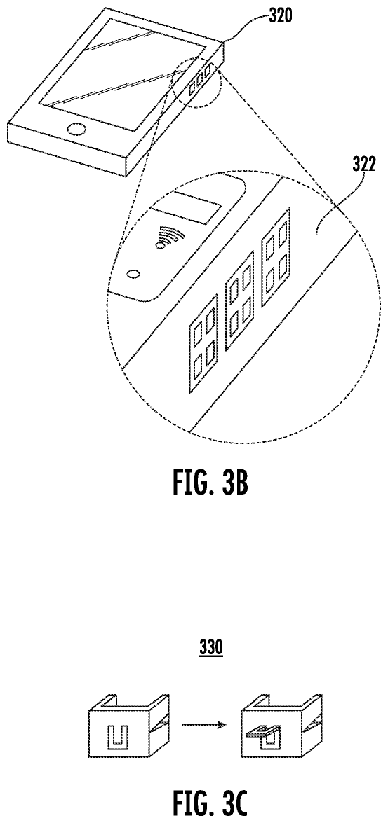

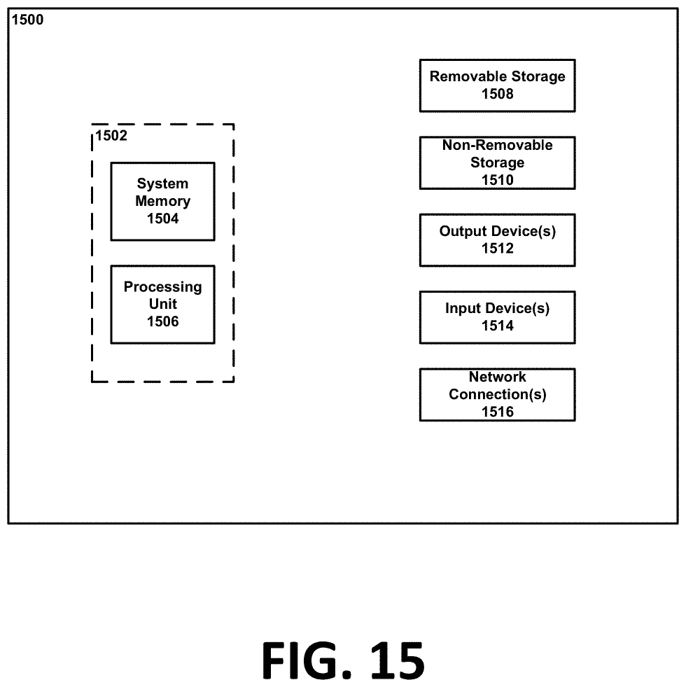

With reference to FIG. 1, a method 100 for performing environmental measurements is illustrated. This disclosure contemplates that the method 100 can be performed using the appliance and/or the system shown in FIGS. 2 and 3. Additionally, and as discussed below, logical operations can be performed using a computing device such as computing device 1500 shown in FIG. 15. At step 102, a first signal is received from an appliance (e.g., system 200 shown in FIG. 2 or magic-wand appliance 300 shown in FIG. 3) that is configured to measure an environmental condition. The first signal can be a signal from an orientation sensor such as an inertial measurement unit (IMU), a sensor for collecting data suitable for biometrics, or any other sensor suitable for determining the identity and/or intent of the user.

Throughout the present disclosure, “identity” is used to refer to an individual user (e.g., a person), distinct from any other user, and implementations described herein can determine that a user of the appliance/system is a specific person (i.e. determine the identity of the user). Furthermore, it is contemplated that determining the identity of a user can be part of the process of authenticating the user; for example, as a preliminary step in the process of asserting authorization for the user. That is, authentication of identity is used to establish that a user is an authorized user by determining a user of the appliance/system's identity, and based on that identity determining whether that user is authorized to use the appliance/system. It is contemplated that the identity of a user can be determined either partially or completely by recognizing one or more gestures. In either case, a statistical probability for authentication may be assigned to the putative identity of one of more users. This may be used in combination with other statistics to assert or deny authentication. Throughout the present disclosure “intent” can be used to refer to what operation the user of the appliance/system desires the appliance/system to perform. As non-limiting examples, the user's intent can include taking an environmental sample, dispensing reagents, authenticating the user, and any other operation that the appliance/system is configured to perform. It is contemplated that the intent of the user can be determined either partially or completely by recognizing one or more gestures.

At step 104, the first signal can be analyzed to determine an identity and/or intent of the user. In some implementations, the step of analyzing the identity and intent of the user can be performed based on gesture recognition. As a non-limiting example, if the sensor is an IMU the first signal can be acceleration data collected when the user performs a gesture. The first signal corresponding to the gesture can be analyzed to determine the identity of the user based on unique characteristics of the gesture, and the gesture can also be used to determine the user's “intent.” At step 106, an environmental measurement can be initiated based on the intent of the user. The decision to perform an environmental measurement can be conditional on the identity and intent of the user. At step 108, a second signal is received from the appliance, where the second signal includes information related to the environmental measurement.

In some implementations, the method 100 also can include acquiring the environmental sample; for example, a solid, liquid or gas sample. Furthermore, the method 100 can include dispensing a dosage of a medicine or reagent in response to the second signal.

With reference to FIG. 2, a system block diagram representing an implementation of the present disclosure is illustrated. The system 200 can include a communication module 202, a user interface module 204, a computing device 206 (e.g., at least one processor and memory), a first sensor 208, a second sensor 210, a condensing unit 212 and a dispensing unit 214. It should be understood that the system shown in FIG. 2 is provided only as an example. This disclosure contemplates that a system for intentional sensing of environmental conditions can include more or less of the components shown in FIG. 2.

In some implementations, the system 200 can include an appliance. The appliance can include a housing (described below), the first sensor 208, and the second sensor 210. The first and second sensors can be attached to or arranged within the housing as described below. Optionally, the housing is an elongated cylinder, e.g., the appliance is a wand. In some implementations, the computing device 206 is integrated in the appliance. In other implementations, the computing device 206 is remote from the appliance.

The first sensor 208 can be any sensor that can be used to determine identity and/or intent of a user. For example, in some implementations, the first sensor 208 is a sensor configured to collect biometric data. Non-limiting examples of sensors configured to collect biometric data include, but are not limited to, a camera, a fingerprint sensor, a microphone, an accelerometer, a strain gauge, an acoustic sensor, a temperature sensor, or a hygrometer. It should be understood that data collected by such sensors can be analyzed to determine body measurements and/or calculation, which can be used to identify a user. In other implementations, the first sensor 208 is an orientation sensor. Orientation sensors include one or more gyroscopes, accelerometers, magnetometers, or combinations thereof. An inertial measurement unit (IMU) is an example orientation sensor. It should be understood that data collected by such sensors can be analyzed to determine an intent of user.

Some implementations of the present disclosure can include a communication module 202 configured to operably couple with the computing device 206. The communication module 202 can be coupled to the computing device 206 through one or more communication links. This disclosure contemplates the communication links are any suitable communication link. For example, a communication link may be implemented by any medium that facilitates data exchange including, but not limited to, wired, wireless and optical links. For example, the communication module 202 can be a wireless module; for example, a low power Bluetooth transceiver. The communication module 202 can connect to a phone, computer, or any other networked device. Implementations described herein can communicate with or be controlled by mobile devices, apps, social media platforms, and any other suitable software. The communication module 202 can be used for collecting and transferring data to the computing device 206 and/or any other device. Additionally, in some implementations the system 200 can provide users with educational information (e.g. about pollution, associated adverse health effects, and their exposure environment). This educational information can be stored in the computing device 206, and can be either received or updated using the communication module 202.

In some implementations, the system 200 is configured to collect samples of gases, liquids, and/or solids from an environment for analysis by the second sensor 210. The second sensor 210 can be any kind of environmental measurement sensor. Optionally, in some implementations, the second sensor is a consumable sensor, for example, a single-use sensor. Non-limiting examples of types of second sensors include, but are not limited to, Surface-Enhanced Raman Spectroscopy (SERS) sensors, air and fluid born analyte sensors, electrodes, electrical resistance sensors, magnetoencephalography sensors, impedance plethysmography (or impedance phlebography) sensors, thermistors, strain gauges, LVDTs (Linear Variable Differential Transformers), capacitance sensors, ultrasound/acoustic sensors, or other material property (e.g., density, electrical conductivity, viscosity, etc. sensors). In a non-limiting example implementation, the system 200 is configured to perform environmental measurements using one or more sensors 210. One or more users (e.g. members of the same community, residents of the same region, etc.) can operate one or more systems 200 and thereby generate a plurality of environmental measurements. These environmental measurements can be stored and/or transmitted to a remote computing device for analysis. The environmental measurements can be correlated with health-related information. This can allow environmental and health-care scientists, community members, and/or other interested parties to make associations between environmental quality (e.g., pollutant levels) and local health and illness patterns. These associations can allow more optimal responses to health hazards in real-time, including potential municipal, policy, or business responses.

In some implementations, the system 200 can include a location sensor (e.g. a GPS sensor) and the location sensor can be used to associate environmental measurement data with the locations at which the environmental measurement data was acquired.

Some implementations described herein can include a condensing unit 212. The condensing unit 212 can be configured to store the sample such as gases, fluids, or solids. In some implementations, the condensing unit 212 includes a shutter (not shown) that can lock and seal once the actuator (e.g., one activated using the user interface 204) is pressed after sample collection. The shutter can be a one-time opening shutter. Alternatively or additionally, the shutter can be configured to lock and/or seal to protect a sensitive reagent (e.g. a medication) from unauthorized access.

Some implementations described herein can include one or more dispensing units 214. The dispensing unit 214 can be configured to dispense a reagent, medicine, or other substance. The reagent can be a reagent for treating an environmental pollutant, changing the condition of the environment (e.g. adjusting a pH value), treating a human patient (e.g. a pharmaceutical), or any other purpose. Optionally, the type and/or amount of reagent or medicine can be determined based on the measurement obtained by the second sensor 210, e.g., an amount of reagent needed to balance pH or an amount of medicine to treat a patient's condition. As a non-limiting example, the dispensing unit 214 can include one or more locked compartments constructed with thicker perimeters to prevent unwarranted opening, and the locked compartments can include a one or more doses of medication for a potential patient health crisis. The activation of the dispensing unit can be based on the identity and/or intent of the user. As a non-limiting example, the decision to dispense a medication can be conditioned on determining that the user is authorized to dispense the medication (identity) and that the user intends to dispense the medication. Other information can be stored by the system 200, and/or accessed using the communication module 202, and can be used by the computing device 206 to determine whether to dispense. For example, a decision to dispense a medication can be based in part on information in a medical record.

In some implementations, the housing is a modular housing configured to include compartments configured to store samples and/or perform small-footprint biochemical reactions on the samples (i.e. “condense”). The system 200 can also include a dispensing unit 214 configured to dispense a reagent into the environment and/or a medicine to a patient. As a non-limiting example, the dispensing unit 214 can include a reagent designed to treat or remediate an environmental health hazard.

Sensor information and analytics can also be stored in memory associated with the computing device 206, transmitted via the communication module 202, or stored in memory. As described above, the computing device 206 and/or communication module 202, may be located in any part of the appliance.

The appliance can include a housing adapted to include some or all of the modules shown in FIG. 2. The housing can be configured as a robust, and highly adaptable handheld device that can be a platform for sample collection, environmental sensing, health sensing, and/or pharmaceutical delivering. In some implementations, the housing includes modular components, and any or all of the elements shown in FIG. 2 can be modular and/or detachable from the housing.

In some implementations, a system includes a sensing appliance coupled to a mobile device, application, and/or social media platform. Such a system will not only provide a means for collecting important data but also engage and educate members in the community about pollution, associated adverse health effects, and their exposure environment. In addition, by linking the local pollutant measurements taken by community members with health-related information, environmental and health-care scientists can make associations between pollutant levels and local health and illness patterns. These associations will, in turn, allow more optimal responses to health hazards in real-time, including potential municipal, policy, or business responses.

Implementations of the present disclosure can be configured as a “platform” that can provide a system integration mechanism for a variety of sensors—traditional, MEMs, paper-based, and/or nanotechnological—that can be leveraged to perform a variety of environmental measurements (e.g. community-environmental health). Information from these sensors can be processed/combined based on user inputs. In this example, individual sensors (i.e., “sense” function) can be associated with removable/replaceable modules in the platform. Additionally, individual modules can store gas, fluid, or solid (e.g. air, water or soil) (i.e., “condense” function). Alternatively or additionally, individual modules can store and release on command a reagent or medicine (i.e., “dispense” function). In other words, the platform can integrate the sense, condense, and dispense functionality in a single appliance.

FIG. 3A illustrates a wand-shaped appliance 300 according to one implementation of the present disclosure. As shown in FIG. 3A, an implementation of the appliance 300 shaped as a wand can include a plurality of sensors (not shown) that are included in cylindrical sensing units 302. This disclosure contemplates that the sensors can be one or more of the sensors described above with regard to FIG. 2. There are several reasons for providing and optionally selecting amongst a repertoire of sensing choices: (1) there are single-use sensors which user does not wish to squander, (2) there may be sensing data subject to access rights/privacy rights to take in a locale, and/or (3) the sensors may interfere (e.g. RFI, EMI) or have insufficient bandwidth/storage to all be taken at the same time.

One or more of the sensing units 302 can optionally include a shutter (not shown), and the shutter can be activated by a user interface (e.g. a button) or by motion (e.g. detecting motion using a first sensor located in the first sensor module 308). In the implementation illustrated in FIG. 3A, the first sensor is a 6-axis inertial accelerometer, also included in the cylindrical housing 308. Furthermore, the appliance 300 can include a control and communication module 304 including one or more of the modules described above with regard to FIG. 2. As a non-limiting example, the control and communication module 304 can include one or more of the communication module 202, the user interface module 204 (which may include a switch 306) and the computing device 206. In the non-limiting example shown in FIG. 3A, it is contemplated that these components may be located in module 304, which is located on an end of the appliance 300. It is also contemplated that in some implementations of present disclosure different groupings of components can be grouped in different modules.

Implementations of the present disclosure can include individual cylinders within the housing, and the housing can be shaped as an elongated “wand” (e.g. as shown in FIG. 3A) or any other desired shape. The housing can be sized and shaped such that the appliance is hand held. In some implementations of the present disclosure, a user can activate the appliance by a button, switch, or other actuator directly or by moving the appliance through the air. Additionally, some implementations can be activated by one or both of the sensors in response to environmental sensing analytics. For example, a gas sensor may activate the appliance when the environmental concentration of a certain gas reaches a certain threshold. Furthermore, it is contemplated that the appliance may be activated by determining that a specific event has occurred or is occurring based on the output of one or more of the sensors. For example, if the sensor results show that a patient is in need of medical treatment (e.g. experiencing congestive heart failure), a dispensing unit of the appliance may dispense an appropriate treatment (e.g. a correct dose of Beta-blocker pharmaceuticals to treat the congestive heart failure episode). Implementations described herein can also be used for sampling environmental media. As a non-limiting example, some implementations can be used to sample the air for VOCs (volatile organic compounds) or to sample the water for lead.

In some implementations, the environmental sensor (e.g., second sensor 210 shown in FIG. 2) is configured to test food for one or more toxins or allergens (e.g. peanut, gluten). Additionally, in some implementations, the environmental sensor is configured to test for molds, mildews, and other forms of pollutant.

In some implementations the appliance is configured to collect/evaluate samples taken from a patient (e.g. breath, fluids, etc.).

It is also contemplated that the appliance can capture a sample of any pollutant for later analysis in addition to, or instead of, analysis by the sensors described herein. Furthermore, it is contemplated that implementations of the present disclosure can be used for a wide variety of purposes, and the examples described herein are intended only as non-limiting examples.

In some implementations, the environmental sensor (e.g., second sensor 210 shown in FIG. 2) is a lab on a chip that is configured to perform one or more laboratory functions. The lab on a chip can be positioned on one end of the appliance, or in a sample collection chamber.

According to some implementations of the present disclosure, the appliance can be configured as an “air wand” which can be activated by waving, and in response to waving the wand the condensing unit can be opened and closed to collect a sample of air. The second sensor can be configured to measure one or more properties of the air. In some implementations, there are multiple condensing units, each with one or more sensors. In these implementations, the appliance can capture multiple samples. Additionally, one or more dispensing units can be included in some implementations. As shown in FIG. 3B, implementations of the present disclosure can include sensing modules associated with slots 322 in a mobile phone 320, and it is contemplated that this can require standardization and/or modification of existing components. Implementations of the present disclosure can be configured or manufactured without modifying existing industry standards while providing a high degree of modularity in function and application. As shown in FIG. 3C, implementations of the present disclosure including sockets formed in a mobile phone may include a clip 330 that can be used to activate the slots 322.

Implementations described herein can implement gesture-based control systems that increase user satisfaction with the appliance. For example, holding and waving a “wand” shaped appliance to sample the environment can be more engaging or desirable to potential users than using conventional control or measurement systems.

Implementations described herein can implement gesture recognition systems, either as the only method of control, or in combination with conventional user interfaces (e.g. buttons, switches, etc.). User interfaces including gesture recognition can be advantageous for different types of users. Users that can benefit from gesture recognition include, for example, users who are unable to distinguish different buttons. Gesture recognition technologies can be more interesting/engaging for users. Gesture recognition has been studied as an interface for appliances, including smart televisions [1]. It is typically accomplished by using single gestures or combinations of gestures [2], [3], that are recognized through algorithms processed on data acquired from wearable sensors, and vision sensors [1], [4], [5], and ECG or EMG signals [6], [7], among others. Common algorithms to compute gesture recognition classification include Dynamic Time Warping [8], [9], [10], [11], [12], Hidden Markov Models (HMM's) [2], [6], [10], [13], [14], [15], and Support Vector Machines (SVM) [16], [17], among others. Accuracies above 90% have been achieved in many of these processes [2], [7], [12], [18], [19], making this acceptable for gesture recognition results. Implementations described herein can implement gesture recognition algorithms including Dynamic Time Warping, Hidden Markov Models, and/or Support Vector Machines, in addition to the gesture recognition technologies described in the present disclosure.

### Example

In one non-limiting example implementation described herein, a system of sensory components incorporated into an all-in-one appliance is configured for use in citizen science. Using task-specific sensors, this device is used for the collection of gas, liquid, and solid samples from an environment. This “magic wand” appliance consists of consumable and non-consumable (long term use) sensors within the wand and uses wireless (e.g., Bluetooth) communication to send information to a receiver, (e.g. a mobile phone). The communications can be sent in real-time.

Specific hand gestures can be used to activate specific sensors or groups of sensors. A user-specific (personalized), customizable set of gestures can be recognized and correctly classified. In the example described herein, the sensor chosen for gesture recognition in this application is a LSM9DS1 9-axis accelerometer/gyroscope/magnetometer. In this study, simple movements were classified accurately 92% of the time using a meta-algorithmic approach with the users' dominant hand. Much lower accuracy was acquired with non-dominant hand direction of the device.

For the activation of this “magic wand”, human-computer interface (HCl) is considered through gesture recognition. Alternatively, activation by button pressing has been used for household appliances for generations but (i) it might be difficult for elderly users who are unable to distinguish different buttons within the control system and (ii) doesn't engage younger users like “waving a wand” might. Gesture recognition has been studied as a form of HCl for activation of various appliances, including smart televisions [1]. It is typically accomplished by using single gestures or combinations of gestures [2], [3], that are recognized through algorithms processed on data acquired from wearable sensors and vision sensors [1], [4], [5], and ECG or EMG signals [6], [7], among others.

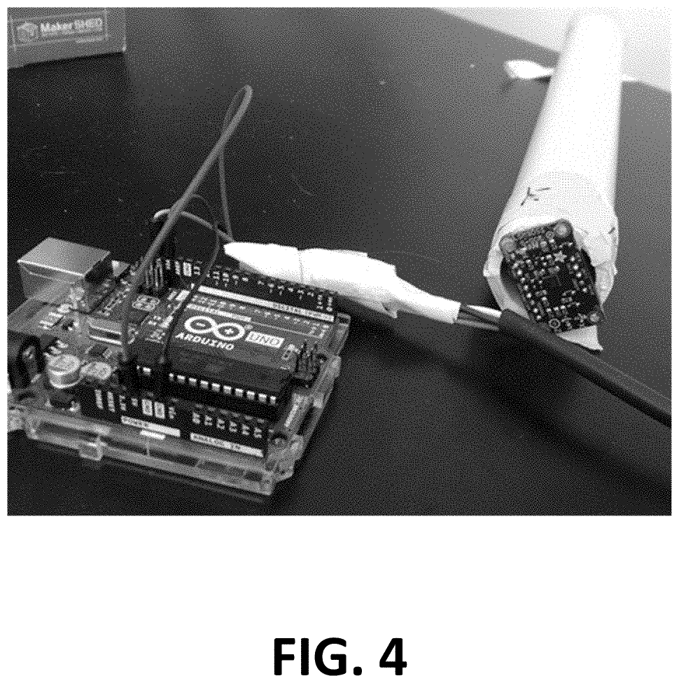

Wearable and handheld sensing options have allowed users to complete gestures without the need for concurrent camera footage. When a movement is made for a gesture, acceleration naturally occurs, and this information can be used to determine how the movement was made along with the path of the extremity. Data from accelerometers, as well as inertial measurement units (IMUs), which include measurements of angular velocity, are very commonly used for gesture recognition techniques, and result in high precision and recall. Activation of the appliance can be triggered by one or more personalized, user-dependent, and customizable gestures. The gestures can be recognized using data acquired from an accelerometer (for example an experimental setup showing an LSM9DS1 9-axis accelerometer/gyroscope/magnetometer connected to an Arduino UNO is shown in FIG. 4)). The Arduino UNO is intended only as a non-limiting example of a general purpose computing device that can be suitable for some implementations described herein. Furthermore, the LSM9DS1 9-axis accelerometer/gyroscope/magnetometer is also intended only as a non-limiting example of a general purpose accelerometer/gyroscope/magnetometer, and the use of other accelerometers, gyroscopes, and/or magnetometers is contemplated by the present disclosure.

This IMU was chosen for the example implementation described herein because it can measure three components of movement: acceleration, angular velocity, and heading [20]. However, it should be understood that the preset disclosure contemplates using combinations of different sensors, and that the LSM9DS1 is intended only as a non-limiting example. The LSM9DS1 described with respect to this example implementation had the following characteristics: (1) the accelerometer has a range of ±2 g to ±16 g; (2) the gyroscope has a range of ±245 to ±2000°/s; and (3) the magnetometer has a range of ±4 to ±16 gauss. The LSM9DS1 is supported by both inter-integrated circuit (I2C) and serial peripheral interface (SPI), making it compatible with not only the Arduino UNO used for prototyping, but most other microcontrollers as well.

Previous studies have found that wearable sensors with a combination of accelerometer and gyroscope data have improved accuracy, precision, and recall [12], [19]. The extra signal from the three gyroscope axes is accountable for this, as it gives information about the users' movements that can give further separation from other movements in algorithms like DTW, HMM, and others. However, for dynamic gestures, it has not been shown that magnetometers provide any improvement to gesture recognition accuracy; therefore, only the accelerometer and gyroscope portions of the 9-axis IMU are used in this study. Other works have also examined differences in movement repeatability between males and females, as well as age differences, and although there have been inconsistent results regarding gender differences in asymmetrical hand movements, it is understood that non-dominant hand movements can be less consistent and result in more error than dominant-hand movements, and that younger users can have less ability to repeat movements consistently [21].

In the non-limiting example described herein, atomic movements (or movements that cannot be decomposed any further) [22] were used for complex gesture recognition. These movements include translational movements (i.e., movements in the x-,y-, and z-directions), as well as rotational movements (i.e., movements in the xy-,yz-, and xz-directions) for a total of six movements. The method of classification for this example is a meta-algorithmic approach that combines an objective function with a support vector machine (SVM), as it has a history of being a strong binary classifier [23], [24], [25]. Previously, this meta-algorithmic approach showed promise as a method of classification. It is also contemplated that implementations of the present disclosure can be applied to therapeutic (e.g. physical therapy) applications; for example, by examining the effects of manipulating the data for translational movements to improve accuracy for both non-dominant handed movements and low-performing dominant-hand movements.

Methods

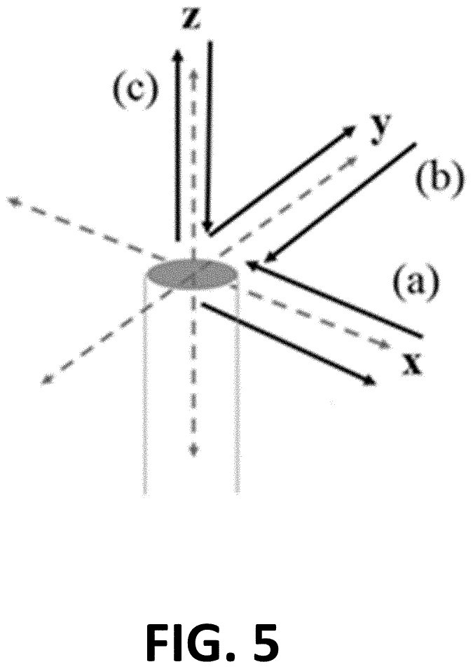

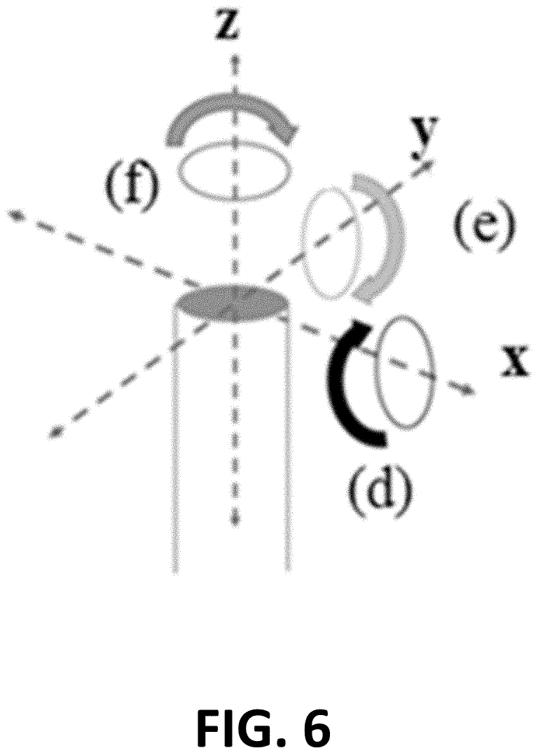

For the example described herein, the 9-axis IMU was connected from the end of a 6-in long PVC pipe (the “wand”) to an Arduino UNO. Five volunteers were asked to move the “wand” in six different movements: three translational movements (FIG. 5), and three rotational movements (FIG. 6). Each movement was completed fifty times for a total of 300 repetitions per user. Accelerometer data measuring acceleration in the x-, y-, and z-directions was stored along with angular velocity data in the roll, pitch, and yaw directions. Samples were measured at a rate of 50 Hz.

In the example described herein, classification was based on a 50% training and 50% testing configuration of the movement set for each user, although it should be understood that the use of other proportions of training and testing for classification is contemplated by the present disclosure. To classify movements, data can be separated into “movement” and “non-movement.” This can be done by adaptive thresholding that can vary from user-to-user. The beginning and ending of each movement can be determined by dividing the data into windows with no overlapping frames. In the non-limiting example implementation the “windows” were 20 ms long. The mean acceleration and angular velocity can be stored. Further, the calibration data acquired during premeasured non-movement can be used to compensate for potential offsets of the sensor, including gravity, and the calibration data can also be stored. Feature extraction was performed based on the movement, non-movement, and calibration data. Movements can be classified using an objective function:

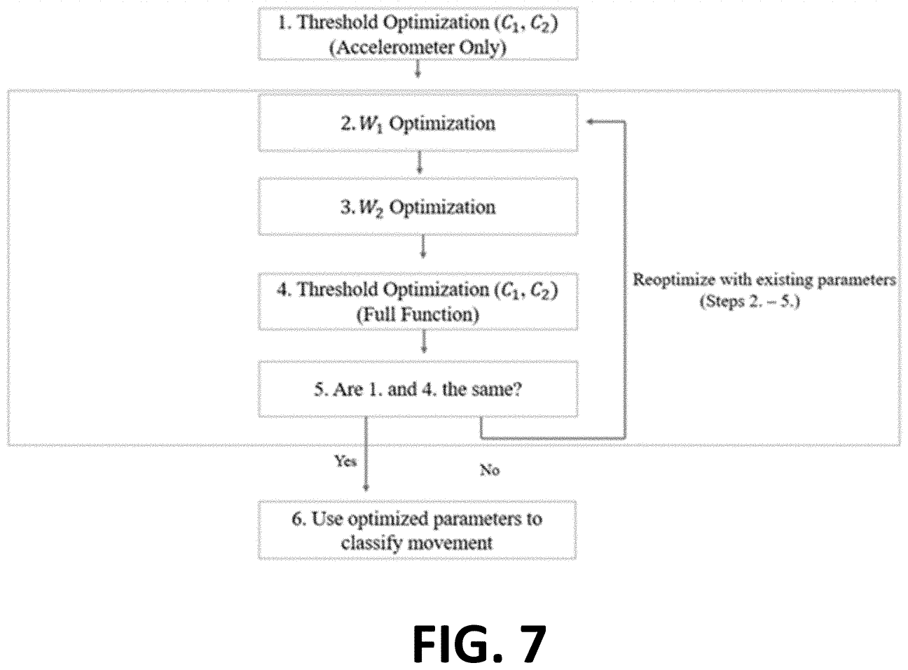

Movements are classified by optimizing an objective function (Eqns. 1-6):

$$\begin{matrix}{J}_{x} = \frac{2 * \vert x - {x}_{o} \vert}{\vert y - {y}_{o} \vert + \vert z - {z}_{o} \vert} + {W}_{2} \frac{\vert p - {p}_{o} \vert + \vert q - {q}_{o} \vert}{\vert r - {r}_{o} \vert} & ( 1 ) \\ {J}_{y} = \frac{2 * \vert y - {y}_{o} \vert}{\vert x - {x}_{o} \vert + \vert z - {z}_{o} \vert} + {W}_{2} \frac{\vert r - {r}_{o} \vert + \vert q - {q}_{o} \vert}{\vert p - {p}_{o} \vert} & ( 2 ) \\ {J}_{z} = \frac{2 * \vert z - {z}_{o} \vert}{\vert x - {x}_{o} \vert + \vert y - {y}_{o} \vert} + {W}_{2} \frac{\vert r - {r}_{o} \vert + \vert p - {p}_{o} \vert}{\vert q - {q}_{o} \vert} & ( 3 ) \\ {J}_{y z} = \frac{\vert y - {y}_{o} \vert + \vert z - {z}_{o} \vert}{2 * \vert x - {x}_{o} \vert} + {W}_{1} \frac{2 * \vert r - {r}_{o} \vert}{\vert p - {p}_{o} \vert + \vert q - {q}_{o} \vert} & ( 4 ) \\ {J}_{x z} = \frac{\vert x - {x}_{o} \vert + \vert z - {z}_{o} \vert}{2 * \vert y - {y}_{o} \vert} + {W}_{1} \frac{2 * \vert p - {p}_{o} \vert}{\vert r - {r}_{o} \vert + \vert q - {q}_{o} \vert} & ( 5 ) \\ {J}_{x y} = \frac{\vert x - {x}_{o} \vert + \vert y - {y}_{o} \vert}{2 * \vert z - {z}_{o} \vert} + {W}_{1} \frac{2 * \vert q - {q}_{o} \vert}{\vert r - {r}_{o} \vert + \vert p - {p}_{o} \vert} & ( 6 )\end{matrix}$$

where x, y, and z are accelerometer data in the x, y, and z directions, respectively; r, p, and q are angular velocity in the roll, pitch, and yaw directions, respectively; x₀, y₀, z₀, r₀, p₀, and q₀ are the respective calibration data for each axes; and W₁ and W₂ are optimized weights determining what relative amount of the gyroscope data will give the best model accuracy for the translational and rotational movements, respectively. Using Eqns. 1, 2, 3, 4, 5, and 6, the maximum of Jₓ, J_{y}, and J_{z} (corresponding to x, y, and z movements, respectively), as well as the maximum of J_{yz}, J_{xz}, and J_{xy} can determine the resulting classified movement by the algorithm. The optimization of the parameters for the objective function is shown in FIG. 7.

To improve accuracy, data manipulation can be applied through projecting the translational movement data onto the respective axis in which the movement was made. This can be done by finding the mean amount of acceleration data in the x-, y-, and z-directions throughout each respective movement, normalizing each vector, and placing it into a matrix (Eqn. 7):

$$S = [ \begin{matrix}{x}_{m , x} & {y}_{m , x} & {z}_{m , x} \\ {x}_{m , y} & {y}_{m , y} & {z}_{m , y} \\ {x}_{m , z} & {y}_{m , y} & {z}_{m , z}\end{matrix} ] \qquad ( 7 )$$

where x_{m,x}, y_{m,x}, and z_{m,x} are the mean accelerometer data for an x movement; x_{m,y}, y_{m,y}, and z_{m,y} are the mean accelerometer data for a y movement; and x_{m,z}, y_{m,z}, and z_{m,z} are the mean accelerometer data for a z movement, respectively. This matrix can be acquired from the training set movement data, and applied onto the test set by using matrix multiplication of the inverse of the normalized matrix by the new movement data (Eqn. 8):

A=S⁻¹M (8)

where S⁻¹ is the inverse of the normalized matrix S, and M is the new movement data. To further analyze the user's movements, the acceleration data can be transformed into distances through integration (Eqn. 9):

distance=Δt²∫_{b}^{e}∫_{b}^{e} acceleration dt² (9)

where Δt is the period between samples, b is the beginning sample of the movement, and e is the ending sample. Data acquired from the three gyroscope axes cannot be similarly decomposed, as they are one integration away from being constants, and therefore they are left unmanipulated.

The distances the wand travels during each movement can be analyzed by plotting the distances in 3-D space, and in this way the data can be visualized before and after it has been shifted by the axis projection. Finally, the number of movements each user made within 30 degrees of each axis can be determined by using cosine similarities between the distance the movement traveled along its path and its respective axis. An example of this is shown (Eqn.10):

$$cos φ = \frac{x \cdot {x}_{0}}{‖ x ‖ ‖ {x}_{0} ‖} \qquad ( 10 )$$

where x₀ is the x-axis. Using Eqns. 7-10, it is possible to visualize the data in order to better understand how to improve the results of the algorithm, as well as to determine if shifting the data to the respective axis that the user is moving on will improve the accuracy for the translational movements with this algorithm.

Results

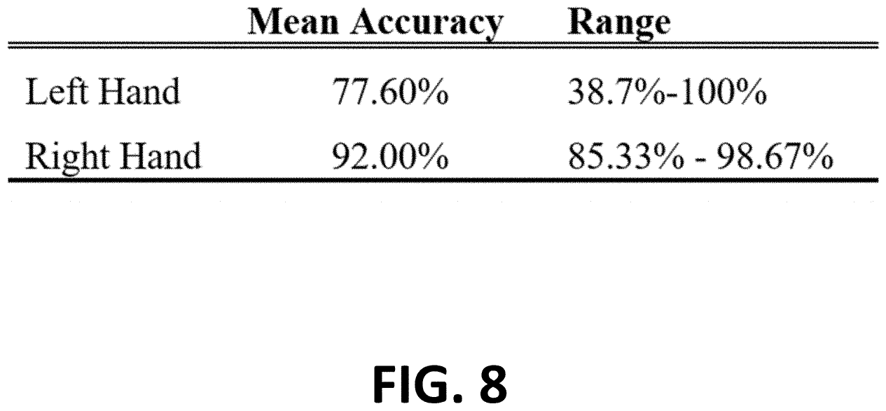

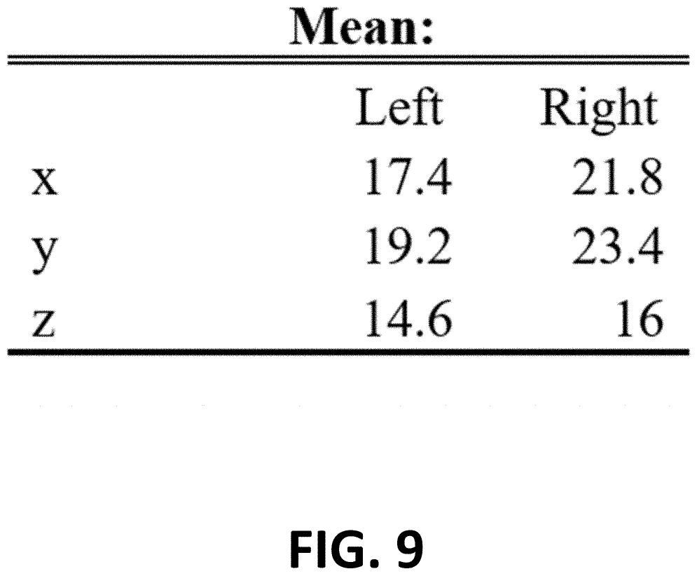

Results of the objective function algorithm (Eqns. 1-6) combined with an SVM are shown in FIG. 8. All five participants are right-handed. For further visualization of the effects of the axis shift (Eqns. 7 and 8), accelerometer data was converted to distance (Eqn. 9) and plotted before and after shifting occurred. As noted above, the use of any suitable accelerometer, gyroscope, magnetometer, or combination of the three is contemplated by the present disclosure, and the LSM9DS1 9-axis accelerometer/gyroscope/magnetometer is intended only as a non-limiting example. For further analysis of the translational movements, the mean number of movements made within 30 degrees of each axis (Eqn. 10) for both left- and right-handed gestures is shown (FIG. 9). The use of other classifiers, including hybrid, ensemble, consensus, or combinatorial approaches is also contemplated by the present disclosure.

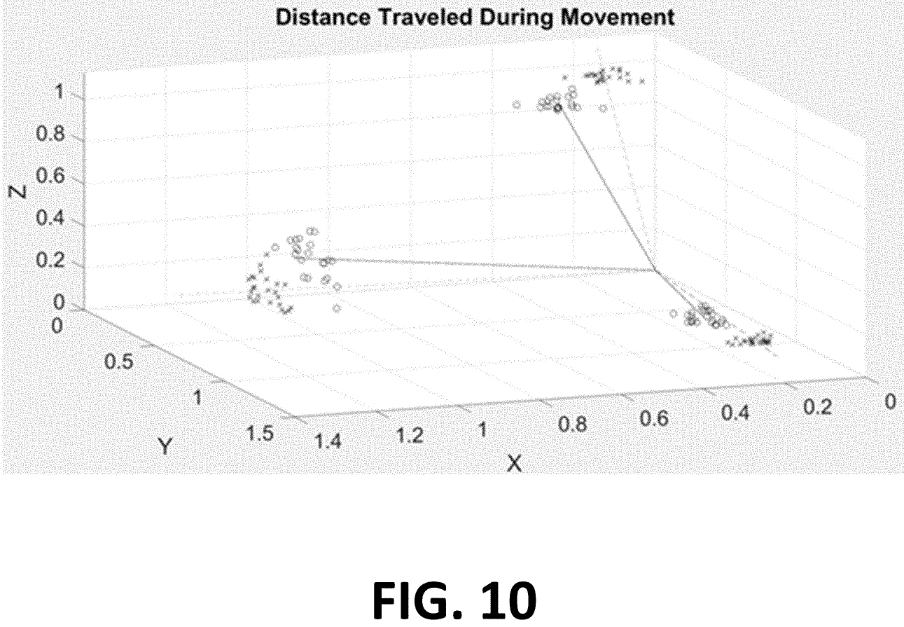

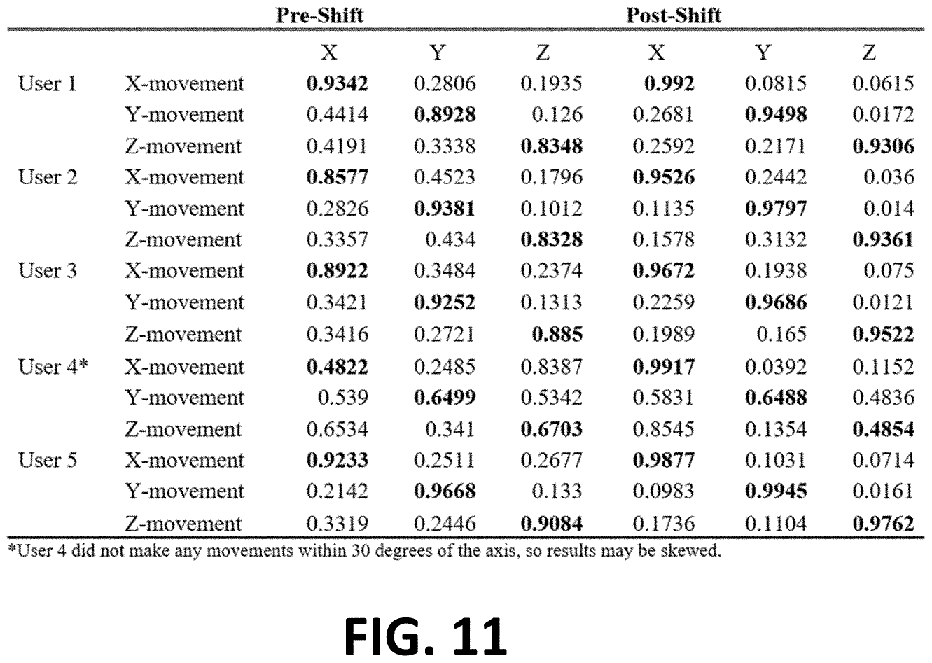

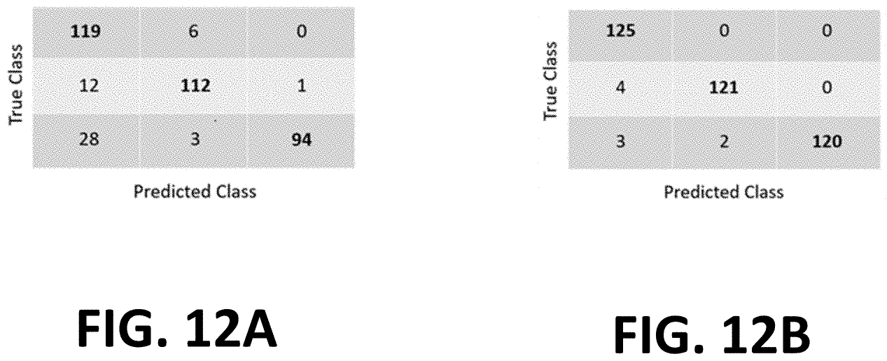

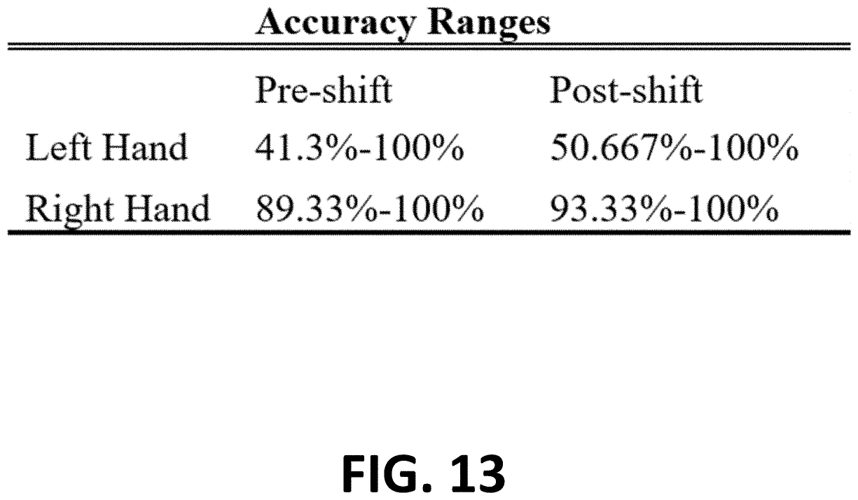

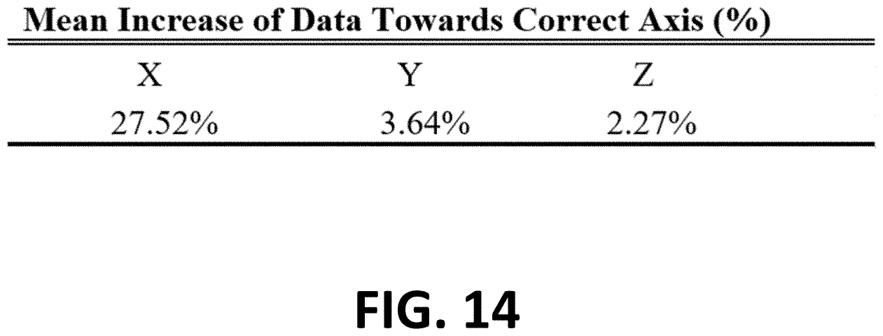

For illustration, a line-of-best-fit was created using a built-in search function in MATLAB known as fminsearch, which optimizes the line to find minimum error between points (FIG. 10). It is contemplated by the present disclosure that other methods of data analysis or visualization can be applied. For example, the line of best fit can be calculated using any suitable algorithm, including linear regression. The mean distance traveled by the movement in each axis before and after the data manipulation (FIG. 11) further quantifies the effect of the data shift. The result of shifting the axis on the accuracy of the translational movements is shown in FIG. 13. The result of shifting the axis on the accuracy of the translational movements is shown in FIG. 14. In the experimental data illustrated in Table 5, the data may be skewed by User 4, who did not make any movements within 30 degrees of any respective axis during the test. An analysis of variance (ANOVA) was run on the ranges of accuracies before and after the axis shift was applied to determine if the change in accuracy between the two methods is significant for both dominant and non-dominant handed movements. The resulting confusion matrices from the axis shift (Eqn. 7 and 8) are shown in FIG. 12.

The optimization of the objective function algorithm (Eqns. 1-6) showed that gyroscope data had no positive effect on the classifications made during this study, which is likely due to the lack of twist in any direction during the movements made during this small proof-of-concept study. The mean number of movements made outside of 30 degrees of the axis during translational movements show that (i) users in this study were able to make movements more repeatably and accurately with their dominant hand, which is agreeable with previous work [21] and (ii) that the movement in the z-direction was the most difficult to repeat accurately (FIG. 9). The change in accuracy from before and after the axis shift for non-dominant hand movements resulted in significant changes in accuracy for the translational movements (F(1,8)=61.47, p<0.001).

The post-movement tracking of data shown in FIG. 10 gives a visualization of the movement for the user to improve their motion, as visualization of movement has been shown to have a positive effect on repeatability and recognition of movements [26]. Data manipulation shown in FIG. 11 shows that the axis shift had the desired effect of shifting the data towards the correct axis for the proposed objective function algorithm. The mean percentage increase of each axis is shown in FIG. 14. This is likely the cause of the significant improvement in the accuracy of the algorithm. Further separation of the data in this way can potentially improve other algorithms that utilize spacing between clusters of datapoints, such as the k-Nearest-Neighbors (kNN) method. The effect of the axis shift also allows for better performance of the objective function algorithm described here, as mathematical separation is achieved with the shift of the data.

Example Computing Device

It should be appreciated that the logical operations described herein with respect to the various figures may be implemented (1) as a sequence of computer implemented acts or program modules (i.e., software) running on a computing device (e.g., the computing device described in FIG. 15), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 15, an example computing device 1500 upon which the methods described herein may be implemented is illustrated. It should be understood that the example computing device 1500 is only one example of a suitable computing environment upon which the methods described herein may be implemented. Optionally, the computing device 1500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 1500 typically includes at least one processing unit 1506 and system memory 1504. Depending on the exact configuration and type of computing device, system memory 1504 may be volatile (such as random access memory (RAM)), non-volatile (such as read-only memory (ROM), flash memory, etc.), or some combination of the two. This most basic configuration is illustrated in FIG. 5 by dashed line 1502. The processing unit 1506 may be a standard programmable processor that performs arithmetic and logic operations necessary for operation of the computing device 1500. The computing device 1500 may also include a bus or other communication mechanism for communicating information among various components of the computing device 1500.

Computing device 1500 may have additional features/functionality. For example, computing device 1500 may include additional storage such as removable storage 1508 and non-removable storage 1510 including, but not limited to, magnetic or optical disks or tapes. Computing device 1500 may also contain network connection(s) 1516 that allow the device to communicate with other devices. Computing device 1500 may also have input device(s) 1514 such as a keyboard, mouse, touch screen, etc. Output device(s) 1512 such as a display, speakers, printer, etc. may also be included. The additional devices may be connected to the bus in order to facilitate communication of data among the components of the computing device 1500. All these devices are well known in the art and need not be discussed at length here.

The processing unit 1506 may be configured to execute program code encoded in tangible, computer-readable media. Tangible, computer-readable media refers to any media that is capable of providing data that causes the computing device 1500 (i.e., a machine) to operate in a particular fashion. Various computer-readable media may be utilized to provide instructions to the processing unit 1506 for execution. Example tangible, computer-readable media may include, but is not limited to, volatile media, non-volatile media, removable media and non-removable media implemented in any method or technology for storage of information such as computer readable instructions, data structures, program modules or other data. System memory 1504, removable storage 1508, and non-removable storage 1510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1506 may execute program code stored in the system memory 1504. For example, the bus may carry data to the system memory 1504, from which the processing unit 1506 receives and executes instructions. The data received by the system memory 1504 may optionally be stored on the removable storage 1508 or the non-removable storage 1510 before or after execution by the processing unit 1506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### REFERENCES

[1] Shiguo Lian, Wei Hu, Kai Wang. “Automatic User State Recognition for Hand Gesture Based Low-Cost Television Control System,”, in IEEE Transactions on Consumer Electronics, IEEE, 2014.
[2] Kirsti Grobel, Marcell Assan. “Isolated Sign Language Recognition using Hidden Markov Models,” in IEEE International Conference on Systems, Man, and Cybernetics. Computational Cybernetics and Simulation, IEEE, 1997.
[3] Kirsti Grobel, Marcell Assan. “Isolated Sign Language Recognition using Hidden Markov Models,” in IEEE International Conference on Systems, Man, and Cybernetics. Computational Cybernetics and Simulation, IEEE, 1997.
[4] Boon Giin Lee, Su Min Lee. “Smart Wearable Hand Device for Sign Language Interpretation System With Sensors Fusion,” in IEEE Sensors Journal, IEEE, February 2018.
[5] Simon Fothergill, Helena Mentis, Pushmeet Kohli, Sebastian Nowozin. “Instructing People for Training Gestural Interactive Systems,” ACM, 2012.
[6] Xu Zang, Chen Xiang, Wen-hui Wang, Jihai Yang. “Hand gesture recognition and virtual game control based on 3D accelerometer and EMG sensors,” International Conference on Intelligent User Interfaces, ACM Press, New York, N.Y., pp. 401-406, 2009.
[7] Xu Zhang, Xiang Chen, Yun Li, Vuokko Lantz, Kongqiao Wang, Jihai Yang. “A Framework for Hand Gesture Recognition Based on Accelerometer and EMG Sensors” IEEE Transactions on Systems, Man, and Cybernetics, IEEE, pp. 1064-1076, 2011.
[8] G. A. ten Holt, M. J. T. Reinders, E. A. Hendriks. “Multi-Dimensional Dynamic Time Warping for Gesture Recognition,” in Thirteenth Annual conference of the Advanced School for Computing and Imaging, June 2007.
[9] Jiayang Liu, Zhen Wang, Lin Zhong, Jehan Wickramasuriya, Venu Vasudevan. “uWave: Accelerometer-based Personalized Gesture Recognition and Its Applications,” Pervasive and Mobile Computing, Elsevier, 2009.
[10] Rossana Muscillo, Maurizio Schmid, Silvia Conforto, Tommaso D′Alessio. “Early recognition of upper limb motor tasks through accelerometers: real-time implementation of a DTW-based algorithm,” Computers in Biology and Medicine, Elsevier, pp 164-172, 2011.
[11] G. A. ten Holt, M. J. T. Reinders, E. A. Hendriks. “Multi-Dimensional Dynamic Time Warping for Gesture Recognition,” in Thirteenth Annual conference of the Advanced School for Computing and Imaging, June 2007.
[12] Konstantinos Dermitzakis, Alejandro Hernandez Arieta and Rolf Pfeifer. “Gesture recognition in upper-limb prosthetics: A viability study using Dynamic Time Warping and gyroscopes,” in Annual International Conference of the IEEE Engineering in Medicine and Biology Society, IEEE, August 2011.
[13] Mahmoud Elmezain, Ayoub Al-Hamadi, Jorg Appenrodt, Bernd Michaelis. “A Hidden Markov Model-Based Continuous Gesture Recognition System for Hand Motion Trajectory,” in 2008 19th International Conference on Pattern Recognition, IEEE, 2009.
[14] Jie Yang, Yangsheng Xu. “Hidden Markov Model for Gesture Recognition,” Defense Technical Information Center Pittsburgh, Pa., 1994.
[15] Thad Starner, Alex Pentland. “Real-Time American Sign Language Recognition from Video Using Hidden Markov Models,” Motion-Based Recognition, Springer, pp 227-243, 1997.
[16] Ganesh R. Naik, Dinesh Kant Kumar, Jayadeva. “Twin SVM for Gesture Classification Using the Surface Electromyogram,” in IEEE Transactions on Information Technology in Biomedicine, IEEE, December 2009.
[17] Giuseppe Serra, Marco Camurri, Lorenzo Baraldi. “Hand Segmentation for Gesture Recognition in EGO-Vision,” in Workshop on Interactive Multimedia on Mobile & Portable Devices, New York, N.Y., USA, pp. 31-36, ACM Press, 2013.
[18] Rossana Muscillo, Maurizio Schmid, Silvia Conforto, Tommaso D′Alessio. “Early recognition of upper limb motor tasks through accelerometers: real-time implementation of a DTW-based algorithm,” Computers in Biology and Medicine, Elsevier, pp 164-172, 2011.
[19] Kirsti Grobel, Marcell Assan. “Isolated Sign Language Recognition using Hidden Markov Models,” in IEEE International Conference on Systems, Man, and Cybernetics. Computational Cybernetics and Simulation, IEEE, 1997.
[20] “SparkFun 9DoF IMU Breakout—LSM9DS1”, sparkfun.com/products/13284.
[21] Hanneke van Mier. “Developmental differences in drawing performance of the dominant and non-dominant hand in right-handed boys and girls,” in Human Movement Science, Elsevier, October 2006.
[22] Ari Y. Benbasat, Joseph A. Paradiso. “An Inertial Measurement Framework for Gesture Recognition and Applications,” in International Gesture Workshop, pp. 9-20, May 2002.
[23] Kai-Bo Duan, S. Sathiya Keerthi. “Which Is the Best Multiclass SVM Method? An Empirical Study,” in International Workshop on Multiple Classifier Systems, Springer, Berlin, Heidelberg, pp. 278-285, 2005.
[24] Vladimir N. Vapnik. “An Overview of Statistical Learning Theory,” in IEEE Transactions on Neural Networks, IEEE, Location??, September 1999.
[25] Ana Carolina Relena, Andre C. P. L. F. de Carvalho, Joao M. P. Gama. “A review on the combination of binary classifiers in multiclass problems,” in Science and Business Meida, Sprin“ger, 2009.
[26] Alana Elza FontesDa Gamaab, Thiago Menezes Chavesa Lucas Silva Figueiredoa, Adriana Baltar, Ma Meng, Nassir Navab, Veronica Teichrieb, Pascal Fallavollit. “MirrARbilitation: A clinically-related gesture recognition interactive tool for an AR rehabilitation system,”, in Computer Methods and Programs in Biomedicine, Elsevier, October 2016.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A system for measuring environmental conditions, the system comprising:
an appliance comprising:
a housing,
a first sensor, and
a second sensor configured to measure a property of a sample, wherein the first and second sensors are attached to or arranged within the housing; and
a computing device in operable communication with the appliance, wherein the computing device comprises a processor and a memory, the memory having computer-executable instructions stored thereon that, when executed by the processor, cause the processor to:
receive a first signal from the first sensor;
analyze the first signal to determine an identity and an intent of a user; and
initiate an action using the second sensor based on the intent of the user.

2. The system of claim 1, wherein the first sensor is a sensor configured to collect data suitable for biometrics.

3. The system of claim 2, wherein the sensor configured to collect data suitable for biometrics comprises at least one of a camera, a fingerprint sensor, a microphone, an accelerometer, a strain gauge, an acoustic sensor, a temperature sensor, or a hygrometer.

4. The system of claim 1, wherein the first sensor is an orientation sensor.

5. The system of claim 4, wherein the orientation sensor comprises at least one of a gyroscope, an accelerometer, or a magnetometer.

6. The system of claim 1, wherein the second sensor is a consumable sensor.

7. The system of claim 1, wherein the second sensor is at least one of a Surface-Enhanced Raman Spectroscopy (SERS) sensor, an analyte sensor, a magnetoencephalography sensor, an impedance plethysmography sensor, a plurality of electrodes, a strain gauge, a thermistor, a linear variable differential transformer (LVDT), a capacitance sensor, or an acoustic sensor.

8. The system of claim 1, wherein the sample is a solid, a liquid, or a gas.

9. The system of claim 1, wherein the memory has further computer-executable instructions stored thereon that, when executed by the processor, cause the processor to receive a second signal from the second sensor.

10. The system of claim 1, wherein the appliance further comprises a dispensing unit configured to dispense a dosage of a medicine or an amount of reagent, wherein the dispensing unit is attached to or arranged within the housing.

11. The system of claim 10, wherein the memory has further computer-executable instructions stored thereon that, when executed by the processor, cause the processor to: receive a second signal from the second sensor; and dispense the dosage of the medicine or the amount of reagent in response to the second signal.

12. The system of claim 10, wherein the dispensing unit comprises a locking mechanism.

13. The system of claim 1, wherein the first signal comprises movement data.

14. The system of claim 13, wherein the movement data comprises a plurality of anatomic movements.

15. The system of claim 13, wherein the movement data comprises at least one of acceleration, angular velocity, or heading information.

16. The system of claim 13, wherein analyzing the first signal to determine an identity and an intent of a user comprises applying a gesture algorithm to the first signal.

17. The system of claim 16, wherein the gesture algorithm is a Dynamic Time Warping (DTW) algorithm, a Hidden Markov Model (HMM) algorithm, or a Support Vector Machine (SVM).

18. The system of claim 1, wherein the housing is an elongated cylinder.

19. The system of claim 1, wherein the housing is comprised of a plurality of modular sections, each of the first sensor and the second sensor is attached to or arranged within a respective modular section housing.

20. The system of claim 19, wherein the respective modular section that houses the second sensor is configured to store the sample.

21. The system of claim 20, wherein the respective modular section that houses the second sensor is further configured to contain a reaction involving the sample.

22. The system of claim 1, further comprising a wireless transceiver configured to operably couple the appliance and the computing device.

23. The system of claim 1, wherein the appliance further comprises a location sensor.

24. An appliance for measuring environmental conditions, the appliance comprising:
a housing;
a first sensor;
a second sensor configured to measure a material property of a sample; and
a wireless transceiver in operable communication with the first sensor and the second sensor, wherein the wireless transceiver is configured to operably couple with a remote computing device, and wherein the first sensor, the second sensor, and the wireless transceiver are attached to or arranged within the housing.

25-39. (canceled)

40. A method, comprising:
receiving a first signal from an appliance, the appliance being configured to measure an environmental condition;
analyzing the first signal to determine an identity and an intent of a user;
initiating an environmental measurement of an environmental sample based on the intent of the user; and
receiving a second signal from the appliance, the second signal comprising information related to the environmental measurement.

41-51. (canceled)
